(19) United States
(12) Patent Application Publication
(10) Pub. No.: US 2026/0063584 A1
(43) Pub. Date: Mar. 5, 2026

(54) HEAVY METAL SENSING USING CARBON FIBER ELECTRODE

(71) Applicants: Board of Trustees of Michigan State University, East Lansing, MI (US); Fraunhofer USA, Inc., Plymouth, MI (US)

(72) Inventors: Wen LI, East Lansing, MI (US); James Siegenthaler, Holt, MI (US); G M Hasan Ui Banna, East Lansing, MI (US); Tumi Olason, Nordhavn (DK); Mohammad Kafi Kangi, East Lansing, MI (US)

(73) Assignees: Board of Trustees of Michigan State University, East Lansing, MI (US); Fraunhofer USA, Inc., Plymouth, MI (US)

(21) Appl. No.: 19/314,265

(22) Filed: Aug. 29, 2025

Related U.S. Application Data
(60) Provisional application No. 63/688,939, filed on Aug. 30, 2024.

(51) Int. Cl. G01N 27/30; G01N 1/38; G01N 27/28; G01N 33/24

(52) U.S. Cl. CPC G01N 27/308; G01N 1/38; G01N 27/283; G01N 27/301; G01N 33/245

(57) ABSTRACT

A carbon fiber electrode apparatus is provided. In another aspect, a method of manufacturing a carbon fiber electrode system is provided. A further aspect includes a method of using a carbon fiber electrode system to sense heavy metal in a fluid and/or soil.

### CROSS-REFERENCE TO RELATED APPLICATION

This application claims priority to U.S. Provisional Application No. 63/688,939, filed on Aug. 30, 2024, which is incorporated herein by reference.

### GOVERNMENT RIGHTS

This invention was made with government support under contract 2226500 awarded by the National Science Foundation. The government has certain rights in the invention.

### BACKGROUND

The present disclosure relates generally to sensors and, more particularly, to a heavy metal sensor using a carbon fiber electrode.

Conventional techniques for heavy metal detection including atomic absorption spectrometry (AAS), atomic fluorescence spectroscopy (AFS), inductively coupled plasma atomic emission spectrometry (ICP-AES), and mass spectrometry (ICP-MS) are generally accurate and some of these methods are highly sensitive with part per trillion (ppt) detection limits. However, such methods are not practical for in-situ monitoring outside of a laboratory due to their sensitivity to mechanical vibration, calibration requirements, bulkiness, power consumption, and operating costs. Additionally, they are laborious and complex to use and require specifically trained personnel.

Compared to conventional methods, electrochemical sensors have the advantages of reduced operational expenses, minimal power consumption, enhanced sensitivity, ease of operation, rapid analysis, portability, and applicability for field monitoring of environmental samples. In particular, anodic stripping voltammetry (ASV) detects heavy metals at the sub-part per billion (ppb) level. Liquid Hg is one of the most common electrode materials used for ASV heavy metal detection due to its ability to form homogenous, liquid metal amalgams. However, due to Hg's toxicity and environmental contamination concerns, solid metal electrodes such as bismuth (Bi), gold (Au), and platinum (Pt) have been studied by researchers as alternative electrode materials. But problematically, alloys may form between the analyte and electrode of metal electrodes during the metal deposition process. As soon as metal ions are deposited, the surface properties of the electrode material change, and it is impossible to generate a defect-free pristine surface with this traditional approach. This causes variable metal measurements which negatively influence repeatability.

In other conventional systems, carbon nanotubes (CNTs), graphene, graphene-oxides and glassy carbons have been used to detect heavy metals. However, these devices rely on sophisticated microfabrication facilities to form microfabricated electrodes, which renders the device fabrication both complex and costly. One such device is disclosed in U.S. Pat. No. 11,307,163 entitled “Carbon Nanotube Based Reference Electrodes and All-Carbon Electrode Assemblies for Sensing and Electrochemical Characterization” which issued to Alvarez, et al., on Apr. 19, 2022. This patent is incorporated by reference herein. It is notable that the Alvarez patent teaches fabricating the electrodes in micro-scale for use with very small water and biofluid volumes, and with the electrodes arranged in a triangular cross-sectional orientation as is illustrated in its FIG. 5A, which leads to poor sensing results.

### SUMMARY

In accordance with the present invention, a carbon fiber electrode apparatus is provided. In another aspect, a method of manufacturing a carbon fiber electrode system is provided. A further aspect includes a method of using a carbon fiber electrode system to sense heavy metal in fluid (such as water) and/or soil.

Another aspect of the present sensor apparatus includes: a sample reservoir including a soil inlet; a buffer reservoir; at least one pump connected to the reservoirs; a mixer connected to the pump(s); a housing defining an enclosed cavity configured to receive a soil and buffer mixture from the sample reservoir and a liquid from the buffer reservoir; a wall removably attached to the housing; a potentiostat coupled to a working electrode removably secured to the wall by a first fastener; a reference electrode removably secured to the wall by a second fastener; a counter electrode removably secured to the wall by a third fastener; and distal ends of the electrodes being located internally in the cavity and configured to be in contact with the mixture. A further aspect of the present sensor apparatus includes a heavy-metal sensing, working electrode which includes: elongated carbon fibers configured in a bundle with a laser-cut sensing end; at least one of the carbon fibers having a diameter of 0.94 mm to 0.28 mm; and an insulating polymer securing together the carbon fibers, each of which includes 50 to 16,300 carbon fiber strands, and more preferably 1,450—7 μm diameter carbon fiber strands in a 0.28 mm fiber to 16,300—7 μm. diameter carbon fiber strands in a 0.84 mm fiber. In yet another aspect of the present sensor apparatus, a software program used therein includes: a first set of instructions configured to establish a communication interface between a working electrode, a reference electrode, a counter electrode and a user interface; a second set of instructions configured to send input parameters to a potentiostat; a third set of instructions configured to energize at least one pump to flow a soil solution; a fourth set of instructions configured to energize at least one of the electrodes and to detect electrical output data from at least one of the electrodes in contact with the soil solution; a fifth set of instructions configured to use the potentiostat to map the data and automatically generating a graphical map on a user interface; and at least a sixth set of instructions configured to automatically determine a presence of and a type of heavy metal detected.

Another aspect of the present electrode apparatus and method includes: (a) using a bundle of carbon fibers, bonded together as a rod containing 50 to 16,300 carbon fiber strands in an exemplary configuration; (b) masking one or more longitudinally spaced apart, exterior side surfaces of the bundle; (c) subsequently applying or depositing an insulating coating or layer to the exterior of the bundle; (d) removing the mask(s) to expose the uncoated side surfaces of the bundle; (e) optionally, cutting the bundle into longitudinal sections with each have a coated portion and an exposed portion; (f) electrically connecting an end of a bundle section to an electrical circuit including a printed circuit board and a power supply; (g) inserting the exposed surface(s) of the bundle section into a specimen; and (h) using the section of the bundle as an electrode to send a sensed signal to the electrical circuit. Still another aspect includes using a carbon fiber bundle, with an exposed exterior portion and an insulated portion, as an electrode to sense the presence of and/or amount of a heavy metal in water or soil. Furthermore, an aspect of the present apparatus and method includes tailoring an exposed area versus an insulated area on an exterior of a bundled carbon fiber electrode to match a desired electrode signal, a desired impedance output, a desired scan rate, pH of a buffer solution, the desired heavy metal to be sensed, or the like. In one exemplary configuration, a diameter of the carbon fiber and epoxy bundle is 0.25-1.0 mm.

The present carbon fiber electrode is advantageous over conventional devices. For example, the present carbon fiber bundle is easier to handle, easier to see without the need for a microscope, and provides a significantly larger sensing surface area as compared to conventional single fiber use for neurochemical detection. As another example, the present carbon fiber electrode apparatus and method are well suited for low cost and accurate sensing of heavy metals in water or soil, without the need for undesirable surface functionalization of an electrode, without the need for toxic heavy metal coating of an electrode before its use, and without the need for expensive microfabrication in a cleanroom. The present carbon fiber electrode apparatus and method are advantageously accurate, low cost and portable, which is beneficial for field transportation and use for heavy metal sensing. The present carbon fiber electrodes are less expensive, eco-friendly, and easy to fabricate without using a cleanroom fabrication facility. Moreover, the surface of the present carbon fiber electrodes can be made self-renewable by etching the outer surface. This reduces surface fouling and enables prolonged sensing application by using a fresh electrode surface in each measurement. Additional features and benefits will become apparent from the following description and claims, as well as the appended figures.

### BRIEF DESCRIPTION OF THE DRAWINGS

FIG. 1 is a perspective view showing the present sensor apparatus;

FIG. 2 is an elevational view showing a fluidic interface of the present sensor apparatus;

FIG. 3 is an exploded perspective view showing the fluidic interface of the present sensor apparatus;

FIG. 4 is a vertical cross-sectional view showing the fluidic interface of the present sensor apparatus;

FIG. 5 is a horizontal cross-sectional view showing the fluidic interface of the present sensor apparatus;

FIG. 6A is an elevational view showing a rod assembly of the present sensor apparatus;

FIG. 6B is an enlarged and vertical cross-sectional view, taken within circle 6B from FIG. 6A, showing the rod assembly of the present sensor apparatus;

FIG. 7A is a perspective view showing a counting electrode of the present sensor apparatus;

FIG. 7B is a perspective view showing a reference electrode of the present sensor apparatus;

FIG. 7C is a perspective view showing a working electrode of the present sensor apparatus;

FIG. 7D is an enlarged perspective view, taken within circle 7D of FIG. 7C, showing a top portion of the working electrode;

FIG. 8 is a block diagram of the present sensor apparatus;

FIG. 9A is a microscopic photograph showing the working electrode of the present sensor apparatus, enlarged at 15 μm;

FIG. 9B is a microscopic photograph showing the working electrode of the present sensor apparatus, enlarged at 100 μm;

FIG. 9C is a microscopic photograph showing the working electrode of the present sensor apparatus, enlarged at 500 μm;

FIG. 10A is a diagrammatic view showing a microfabrication process of the working electrode;

FIG. 10B is an elevational view showing an electrical connection of the working electrode;

FIG. 10C is an elevational view, taken perpendicular to that of FIG. 10B, showing the electrical connection with a sensing tip of the working electrode;

FIG. 10D is a microscopic photograph showing a carbon electrode cut by a scissor, enlarged at 1 mm;

FIG. 10E is a microscopic photograph showing the present working carbon electrode cut by laser, enlarged at 1 mm;

FIG. 11 is a block diagram showing the sensing apparatus including a microprocessor and a remote receiver;

FIG. 12 is a graph showing a cyclic voltammetry comparison of the present sensor apparatus, at 7.4 μm, 0.28 mm and 0.94 mm;

FIG. 13 is a peak current graph of the present sensor apparatus, at 7.4 μm, 0.28 mm and 0.94 mm;

FIG. 14 is an enlarged peak current graph of the present sensor apparatus, at 7.4 μm, 0.28 mm and 0.94 mm;

FIG. 15 is a block diagram showing the present sensor apparatus;

FIG. 16 is a diagram showing a user interface program of the present sensor apparatus;

FIG. 17 is a diagram showing a graphical data display of the present sensor apparatus;

FIG. 18 is a software logic flow diagram of the present sensor apparatus;

FIG. 19 is a software logic flow diagram of the present sensor apparatus, employing a differential pulse anodic stripping voltammetry function; and

FIG. 20 is a perspective view showing an alternate embodiment of a fluidic interchange of the present sensor apparatus.

### DETAILED DESCRIPTION

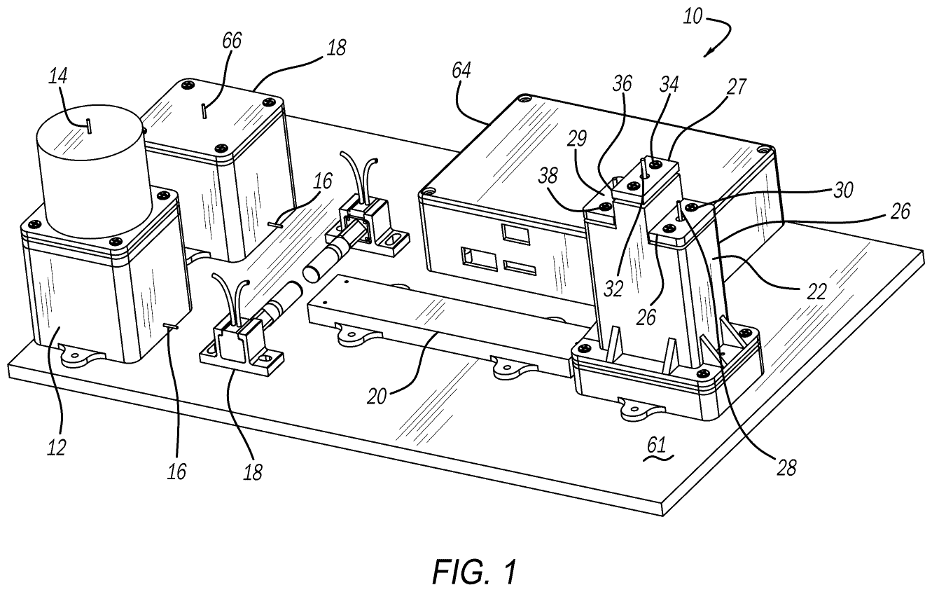

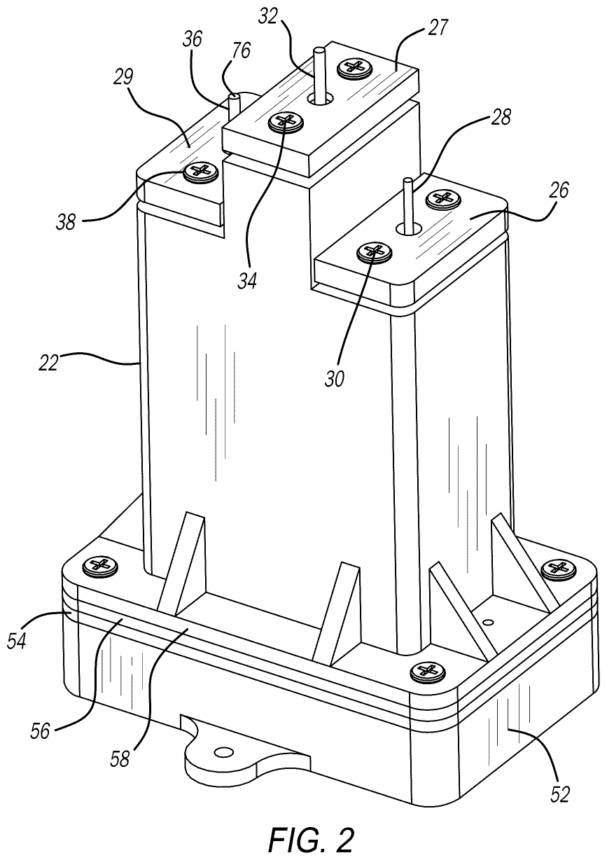

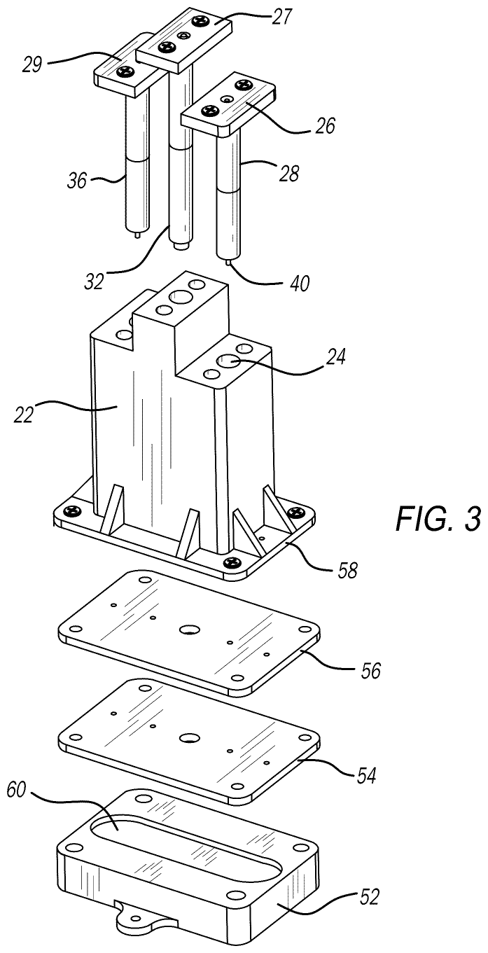

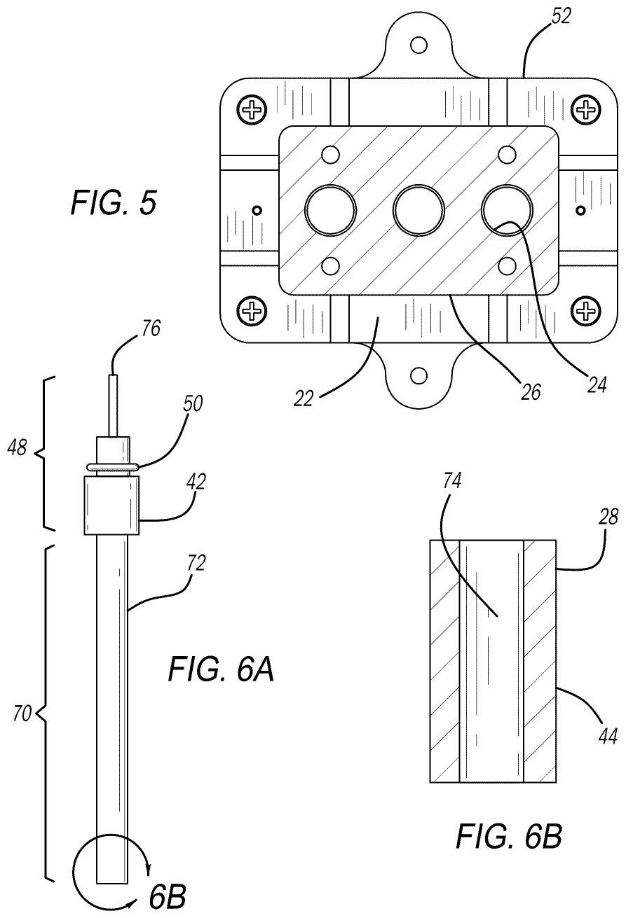

Referring to FIGS. 1-5, 7A-7C and 8, the preferred embodiment of a sensor apparatus 10 is configured for use in monitoring agricultural soil or water contamination of heavy metals including lead, mercury, cadmium and arsenic. The sensor apparatus is preferably a portable and robust unit that can be easily transported and used in the field. Sensor apparatus 10 may be carried by a person from field site to field site, or may be mounted to a vehicle such as to a tractor-pulled plow or disc for an agricultural field, an earth mover or bulldozer at a construction site, a drill at a well boring site, or the like, to test the soil as it is being otherwise worked. It is beneficially enclosed and self-contained so as to withstand and accurately function even with expected environmental dirt and moisture outside of a laboratory.

Alternately, the sample may include a beverage or food, such as baby food, baby formula, milk and the like. Accordingly, the sensor apparatus may continuously test the sample in a real-time, flow through manner as it is being manufactured or processed, or afterwards in random, batch quality testing.

Sensor apparatus 10 includes a sample reservoir 12 that is coupled with a soil inlet 14, where a soil sample enters, and an outlet 16 coupled to a pump 18 where the soil sample is dispersed. The soil sample is preferably a combined dirt and liquid solution. The inlet preferably is an elongated tube that can suck in the soil sample, but may alternately be a chute for manual soil feeding. The sensor apparatus also contains a buffer reservoir 18 with a buffer inlet 66. A liquid buffer solution is fed into the buffer reservoir via a flexible tube or, alternately, a manual filling port, and an outlet 16 is coupled to a second pump 18 where the buffer solution is dispersed.

Pump 18 is connected to a mixer 20 where the buffer sample and soil sample are mixed into a sample mixture via intertwining and arcuate channels. The sample mixture is then pumped into a housing 22, where it flows into and through a basin 60 inside a microfluidic base 52. Microfluidic base 52 has a bottom housing aligner 45 and a top housing aligner 58 with a gasket 56 therebetween. The reservoirs, pumps, mixer and housing components are all mounted on an enlarged and generally flat plate 61, and the components are optionally encased within a removable enclosure or cover attached upon the plate.

Housing 22 also includes an internal cavity 24 where a working electrode 28, a reference electrode 32 and a counter electrode 36 are removably inserted. Working electrode 28 is secured to an upper cap or wall 26, which is removably mounted upon a lower-stepped top portion of housing 22 by a first set of fasteners 30, such as threaded screws. Reference electrode 32 is secured to another upper wall 27, which is removably mounted upon a central and raised top portion of housing 22 by second set of fasteners 34, such as threaded screws. Furthermore, counter electrode 36 is secured to an upper wall 29, which is removably mounted upon another lower-stepped top portion of housing 22 by a third set of fasteners 38, such as threaded screws. Working electrode 28, reference electrode 32 and counter electrode 36 are all co-planar with each other and in that respective order with the reference electrode between the other two; it has been found that such an arrangement provides superior sensing results.

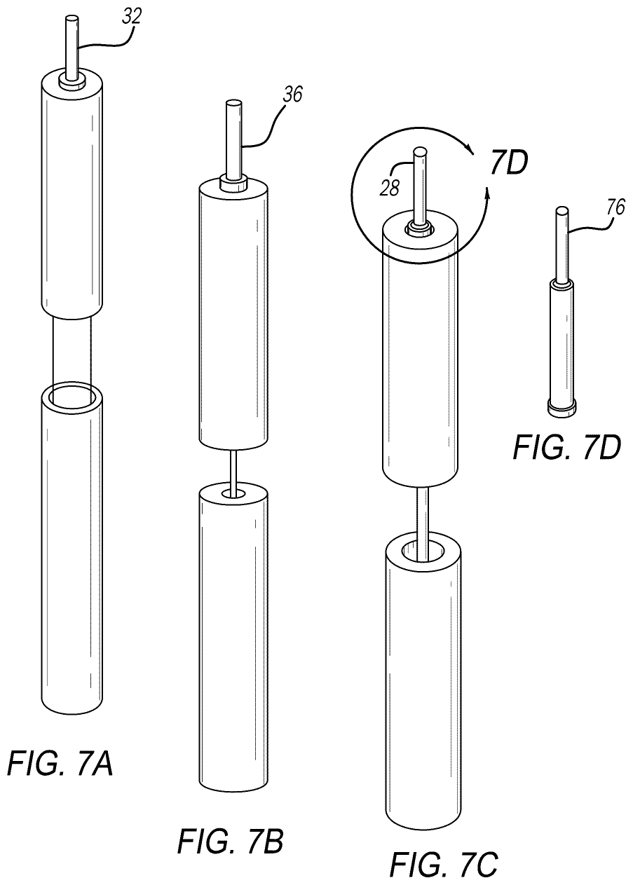

Each electrode is removably secured to its associated upper wall, with a proximal end 76 (see FIG. 7D) of the electrode projecting through an aperture in a center of wall 26. Alternate removable fasteners may be employed, such as bolts, snap-fit clips, pins or the like. While the differing height, removable walls 26, 27 and 29 are preferably of a stepped configuration with the central wall 27 being a greatest distance from the plate, alternate heights and shapes may be employed, although some of the present benefits may not be fully realized.

A potentiostat 64 is coupled to the proximal end of each electrode. Potentiostat 64 includes an operational amplifier (op-amp) which is a voltage-amplifying component, and a signal generation and measurement component that generates applied waveforms and converts the voltage and current observed into measurable output signals or data.

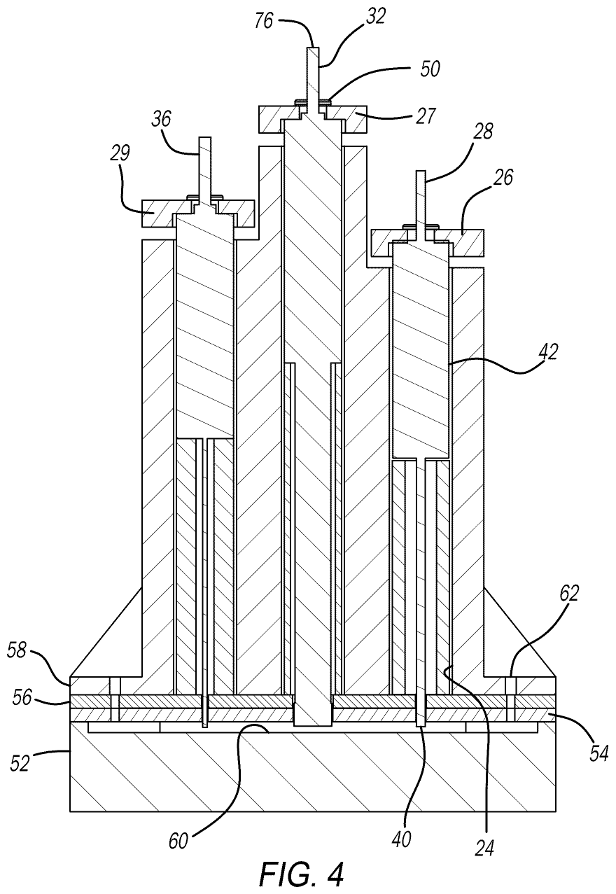

FIGS. 4, 6A, 6B, 7C and 7D illustrate working electrode 28 in greater detail as being in the form of a rod assembly 42 including a top or proximal portion 48 with a rubber or elastomeric seal block 46, and a rubber or elastomeric sealing ring 50 attached thereto. A bottom portion 70 of rod 72 is disposed within an internal and linearly elongated passageway in the housing.

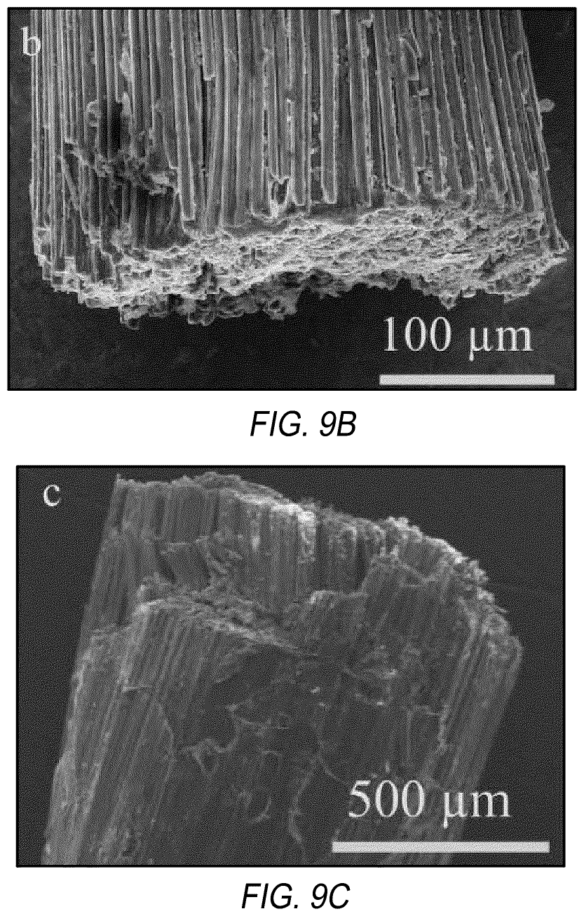

Carbon fibers 74, such as those shown in FIGS. 9A-9C, of the working electrode are bundled together and encapsulated within an insulating polymer 44. Parylene-C material may be used as an outer encapsulation of the carbon fiber bundles, and a polymeric resin can be employed to secure the carbon fibers within each bundle. Ag/AgCl material is preferably employed as reference electrode 32 (which may be obtained from CH Instruments, Inc.). Furthermore, a platinum wire material is preferably used for counter electrode 36.

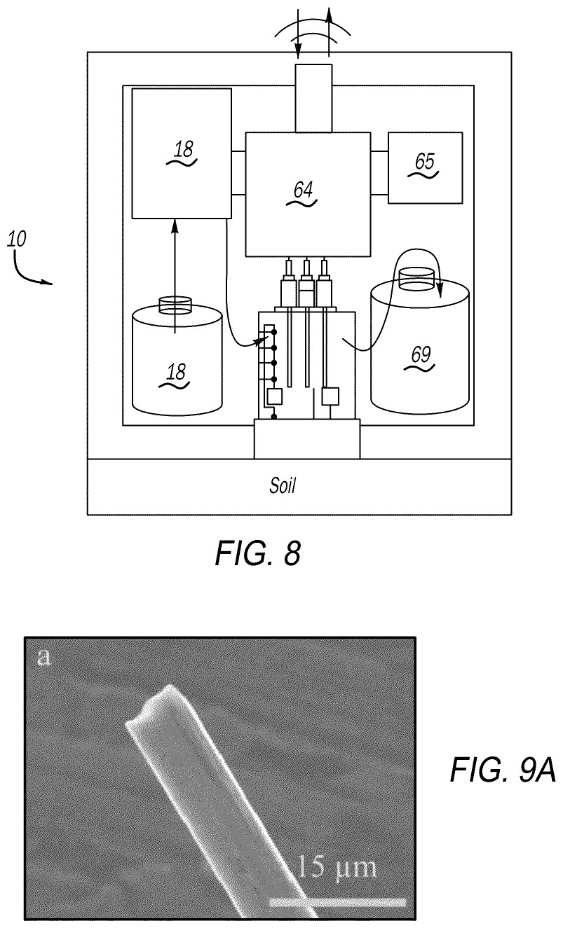

FIGS. 8, 10 and 15 are block diagrams of the working system of sensor apparatus 10. A power source such as a battery 65 is connected to potentiostat 64 which is also connected to pump 18. As previously mentioned, potentiostat 64 is connected to counter electrode 36, reference electrode 32 and working electrode 28. A programmable controller includes a microprocessor 67 which is connected to potentiostat 64 and battery 65. After the electrode output data is collected by the microprocessor, the sample is sent to the waste bottle or container 69. Microprocessor 67 is located within housing 22 and is optionally in communication with a remote receiver 71, such as a portal cellphone and/or central computer controller via a wireless signal such as a Bluetooth, wi-fi, cellular phone tower, satellite or the like, to collect the electrode data and/or display the results. The on-board microprocessor 67 and/or the remote controller may do the calculating, mapping and determination functions.

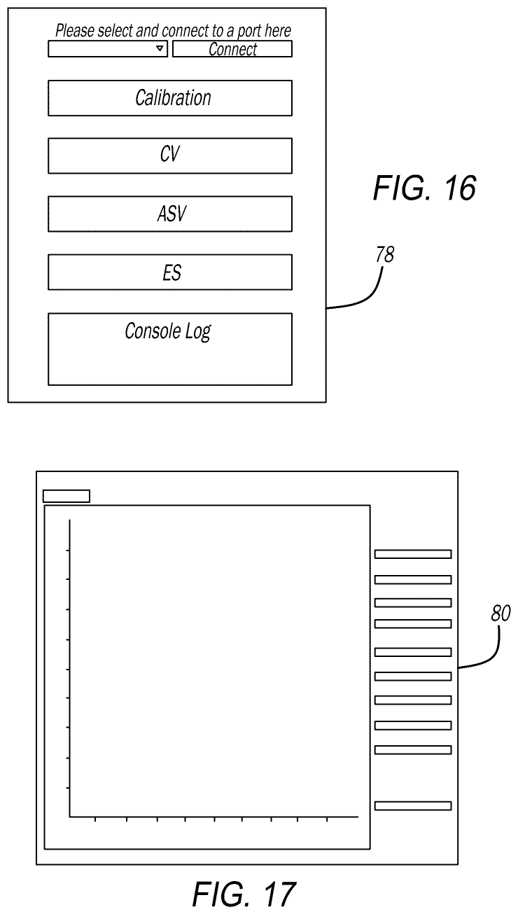

FIG. 16 shows a user interface 78 program including a calibration function and functions for different experiments or tests using the apparatus. Moreover, FIG. 17 depicts an interface data display 80 showing calculated graphs containing mapped output data collected from the electrodes and potentiostat.

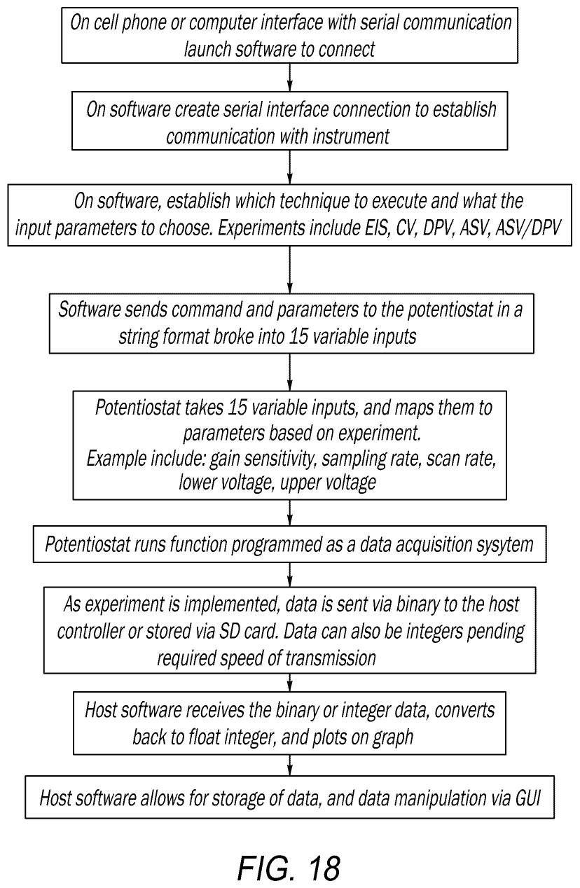

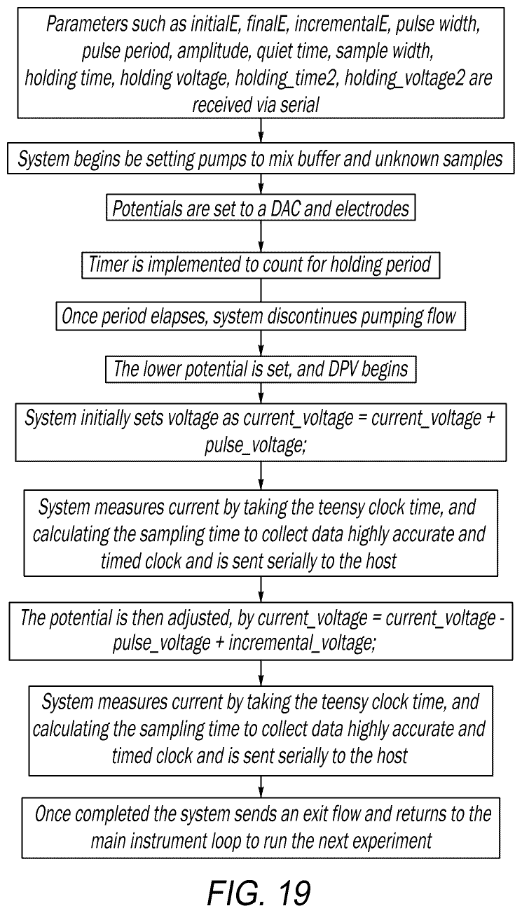

Programmed software instructions, stored in non-transient RAM, ROM or removable memory of the controller and run on the microprocessor 67 and/or the remote controller 71, are illustrated in FIGS. 18 and 19. The FIG. 18 software operation stores data and then maps the data onto the graph displayed in the data display 80. Also, FIG. 19 is a logic flow diagram demonstrating software instructions performing a Differential Pulse Anodic Stripping Voltammetry function (DP-ASV). For example, the software program for the present sensor apparatus includes: (a) a first set of instructions configured to establish a communication interface between the working electrode, reference electrode, counter electrode and a user interface; a second set of instructions configured to send input parameters to a potentiostat; a third set of instructions configured to energize at least one pump to flow a soil solution; a fourth set of instructions configured to energize at least one of the electrodes and to detect electrical output data from at least one of the electrodes in contact with the soil solution; a fifth set of instructions configured to use the potentiostat to map the data and automatically generating a graphical map on a user interface; and at least a sixth set of instructions configured to automatically determine a presence of, a type of and a concentration of heavy metal detected. In an optional arrangement, the software instructions employ parameters including at least one of: finale, increment, pulse width, pulse period, amplitude, quiet time, width, holding time, and/or holding voltage. In another optional arrangement, the software instructions employ functions including at least one of: electrochemical impedance spectroscopy, cyclic voltammetry, differential pulse voltammetry, anodic stripping voltammetry, and/or combined anodic stripping voltammetry with differential pulse voltammetry with the functions being preprogrammed. In yet another optional arrangement, the software instructions employ a current calculated by taking a clock time and sampling the time to collect highly accurate data and sending that data serially to the processor. Moreover, the software instructions of another optional arrangement, calculate and display a graph report based on a point-by-point waveform generation up to 100 kHz. The software code may combine or further separate the programmed instructions.

FIG. 9A shows a SEM image of an exemplary carbon fiber diameter of 7.4 μm for the working electrode, while FIG. 9B shows a SEM image of an exemplary carbon fiber diameter of 0.28 μm for the working electrode, and FIG. 9C shows a SEM image of an exemplary carbon fiber diameter of 0.94 μm for the working electrode. Single AS4 carbon fiber strands with a diameter of 7.4 μm may be obtained from Hexcel Corporation, by way of nonlimiting example, and they have a resistivity of 1.7×10−3 ohm-cm and 94% carbon content. Carbon fiber composite rods with 0.28 mm and 0.94 mm diameters are obtained from CST—The Composites Store, Inc, also by way of nonlimiting example. These composite rods are constructed by binding together multiple strands of T700S carbon fibers (resistivity 1.6×10⁻³ ohm-cm) with a bisphenol epoxy, producing rods with a fiber volume of 63% and 60% for the 0.28 mm and 0.94 mm rods, respectively. FIGS. 9A and 9C illustrate that the thicker fibers are composed of a bunch of thinner carbon fibers, bound together.

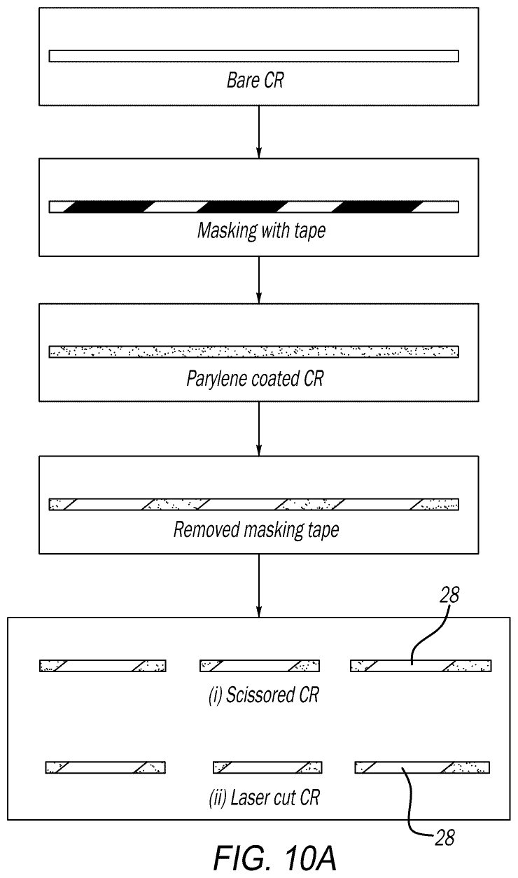

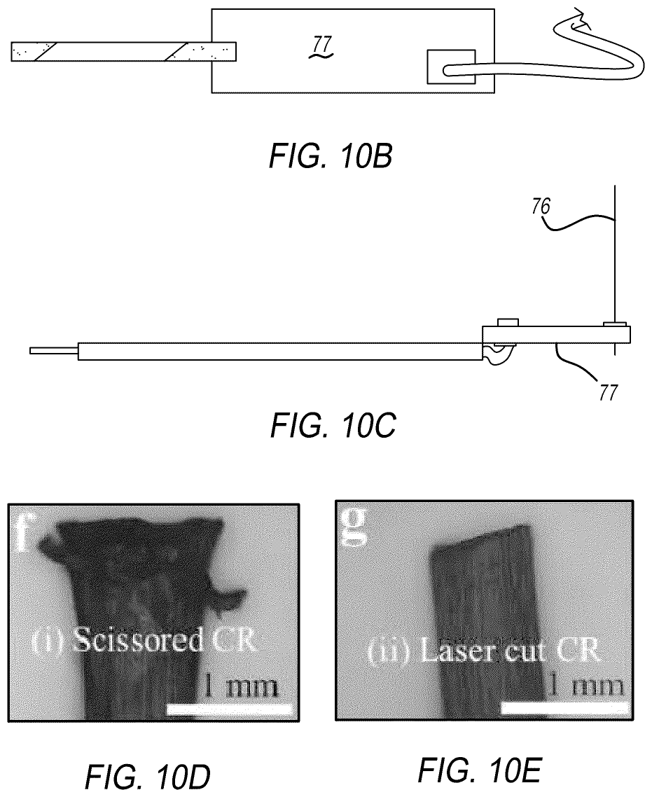

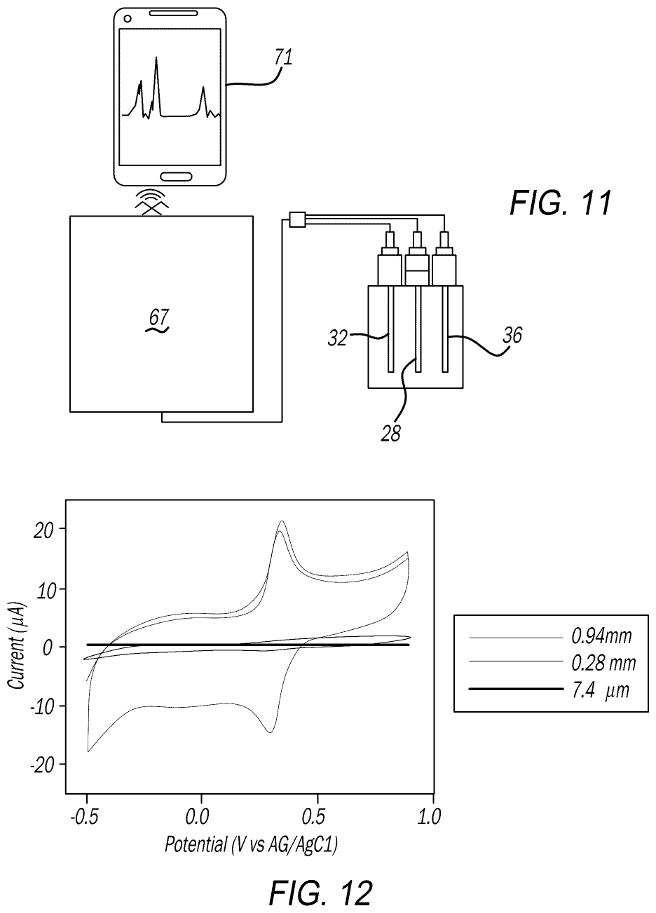

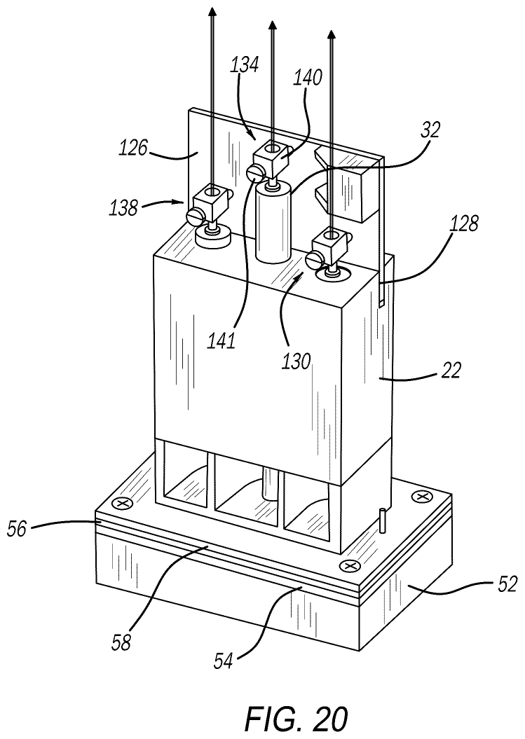

Referring now to FIGS. 10A-10C, a microfabrication process of carbon fibers 74 for one configuration of working electrode 28 are fabricated in an open lab environment using a microfabrication method, using the 0.28 mm and 0.94 mm diameter carbon fibers. The working electrode is fabricated by wrapping the carbon fibers with masking tape so that some parts of the carbon fibers are exposed, and some parts are covered with the tape. Then a 1-20 μm layer of insulating polymer 44 (a non-conductive coating) is conformally deposited over the carbon fibers in open lab. Later, the masking tape is mechanically removed and the working electrode 28 is hand-cleaved or more preferably laser cut so that both sides are exposed and the middle is covered with insulating polymer 44. In a less preferred example, scissors are used to hand-cleave working electrode 28, while in a more preferred example, a femtosecond (FS) laser (such as model Astrella-USP-IK from Coherent Corp.) with a wavelength=800 nm, a frequency=I kHz, and power=5 W, is used to laser cut the fibers. Finally, one exposed side is connected to a printed circuit board (PCB) acting as a wall 26 (see FIG. 20) using a conductive carbon adhesive, while the other is used as a sensing electrode as shown in FIG. 10A. For working electrode 28, the cross-sectional areas of the cleaved fibers are the electrode sensing area. FIGS. 10B and 10C show the fabricated working electrode 28 with proximal sensing tip 76 and an electrical connection 77. FIGS. 10D and 10E show the microscopic image of distal end 40 of the 0.94 mm diameter working electrode 28 and the difference between hand cleaved and laser cut working carbon fibers. It should be noted that the laser cut carbon fibers provides a smoother cutting edge and better sensing performance as compared to the hand cleaved end.

The effective areas of 7.4 μm, 0.28 mm and 0.94 mm Ø carbon fiber electrodes are calculated electrochemically by measuring the response of 0.5 mM K₃[Fe(CN)₆] at varying scan rates of 0.01 V/s, 0.02 V/s, 0.05 V/s, 0.1 V/s, 0.2 V/s, and 0.5 V/s using the Randles-Sevcik equation:

i_{P}=2.69×10⁵n^{3/2}AD^{1/2}Cv^{1/2}

where iₚ is the cathodic peak current, n is the number of transported electrons, A (cm²) is the effective electrochemical surface, D (cm²·s⁻¹) is the diffusion coefficient, C (mol·cm⁻³) is the concentration of redox species, and v (V s⁻¹) is the potential scan rate. The dependency of the anodic peak potentials on the natural logarithm of the potential scan rate is investigated and linear fit lines are generated using the cathodic peak current and the square root of the potential scan rate. Using this method, the effective areas of the 7.4 μm, a 0.28 mm and 0.94 mm diameter working electrode 28 are estimated to be around 2.27×10⁻⁵ cm², 8.57×10⁻³ cm², and 1.23×10⁻¹ cm², respectively.

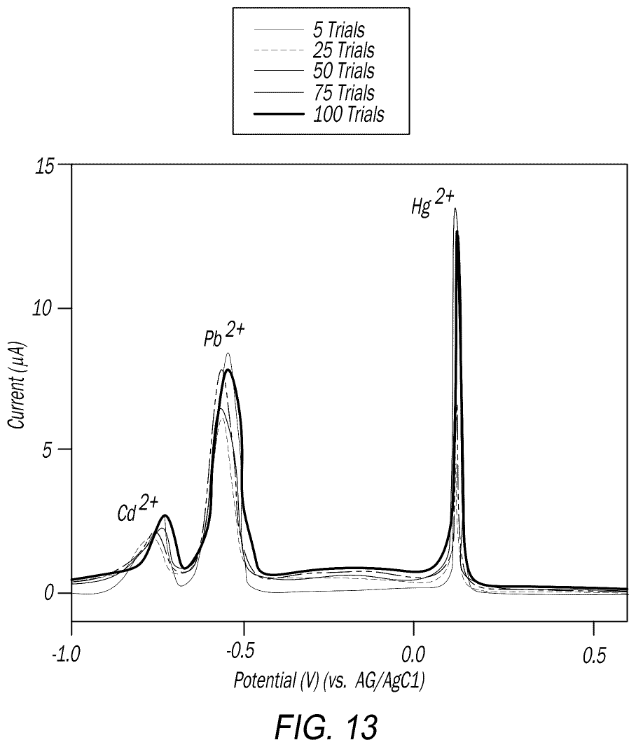

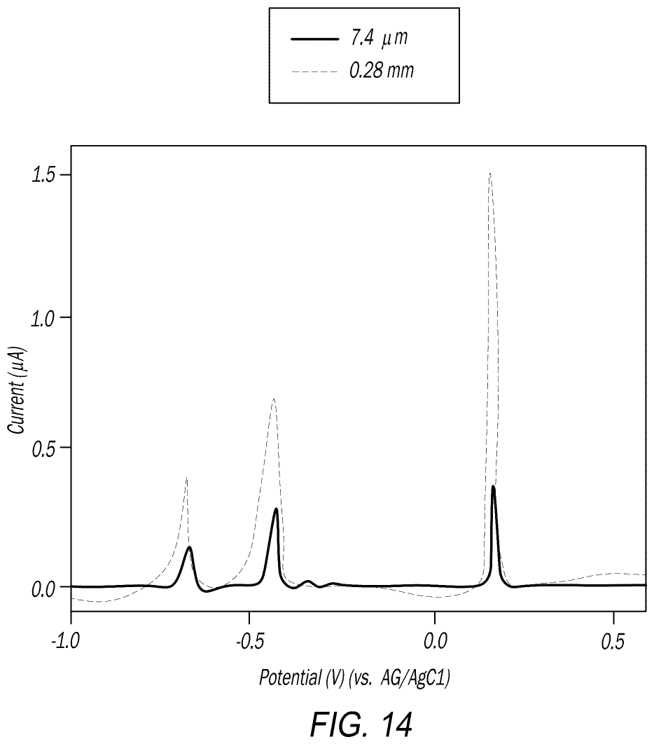

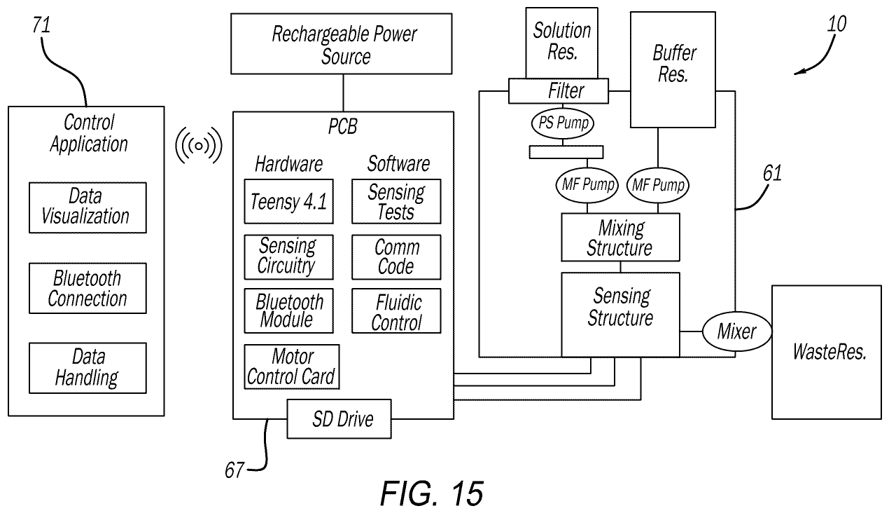

FIG. 12 is a cyclic voltammetry comparison between 7.4 μm, 0.28 mm, and 0.94 mm at CFE in 0.5 mM K₃[Fe(CN)₆] containing 0.1 M KCl with 0.1 M acetate buffer solution (pH=5.0) at scan rate 0.01 Vis (d) EIS spectrum comparison of 7.4 μm, 0.28 mm and 0.94 mm at working electrode 28 in 0.01 M acetate buffer (pH=4.97) solution containing 50 mM NaCl. Furthermore, FIG. 13 depicts peak current of 7.4 μm, 0.28 mm and 0.94 mm at working electrode 28 at 1600 μg/L Cd²⁺, Pb²⁺ and Hg²⁺ respectively in in 0.01 M acetate buffer (pH=4.97) containing 50 mM NaCl. Furthermore, an enlarged graph for the peak current response of heavy metals sensing with 7.4 μm and 0.28 mm at working electrode 28, can be observed in FIG. 14.

Finally, FIG. 20 illustrates a second embodiment of the present sensor apparatus with a printed circuit board wall 126 upwardly projects in a generally vertical manner from housing 22. Notably, a bottom edge of wall 126 removably sits within a matching slot 128 in in an upper surface of housing 22; it may be temporary retained therein by a snap fit attachment, a barbed clip, a threaded screw fastener, a pin or the like. A first fastener 130, a second fastener 134, and a third fastener 138 are soldered or otherwise electrically coupled to conductive printed circuit traces in wall 126. Each block-like body 140 of the fasteners has a central hole through which the proximal end of each electrode 28, 32 and 36 protrude. Each fastener 130, 134 and 138 further includes a screw 141 having a slotted head and a threaded shaft, with the shaft engaging threads of the body such that an inner end of the screw removably traps and holds the distal end of the electrode against an inner surface of the fastener body. This configuration allows each electrode to be removed from their cavity 24 because the fastener is only connected to the housing at distal electrode tip 76. When placed back into cavity 24, the electrode is inserted until the distal end 40 reaches the soil and buffer mixture in basin 60 of microfluidic base 52. Microfluidic base 52 is connected to the bottom of bottom housing aligner 54 and bottom housing aligner 54 is connected to the bottom of gasket 56, which in turn, is connected to the bottom of top housing aligner 58. All of the other features of the present apparatus discussed for the first embodiment are otherwise the same for this second embodiment.

While various features of the present apparatus have been disclosed, it should be appreciated that other variations may be employed. For example, differently shaped or additional housings, cavities, walls and reservoirs may be employed, although various advantages of the present apparatus may not be realized. As another example, different types of fasteners and walls may be used as long as the electrodes are removable for servicing and replacement, but certain benefits may not be obtained. Alternately, other manufacturing processes may be used, however, the present advantages may not be obtained. Features of each of the embodiments and uses may be interchanged and replaced with similar features of other embodiments. Variations are not to be regarded as a departure from the present disclosure, and all such modifications are intended to be included within the scope and spirit of the present invention.

1. A sensor apparatus comprising:
a sample reservoir including a soil inlet and an outlet;
a buffer reservoir including a buffer inlet and outlet;
at least one pump connected to the outlets of the sample reservoir and the buffer reservoir;
a mixer connected to an output of the at least one pump;
a potentiostat coupled to a working electrode removably secured to a wall by a first fastener;
a housing defining an enclosed basin therein configured to receive a soil and buffer mixture from the sample reservoir and a liquid from the buffer reservoir;
a wall removably attached to the housing;
a reference electrode removably secured to the wall by a second fastener;
a counter electrode removably secured to the wall by a third fastener; and
distal ends of the electrodes being located internally in the basin and configured to be in contact with the mixture;
the working electrode comprising a bundle of carbon fibers bonded together as a rod assembly.

2. The sensor apparatus of claim 1, wherein the potentiostat is connected to a programmable controller which is in communication with a remote receiver via a wireless connection to display calculated results from output signals from at least one of the electrodes.

3. The sensor apparatus of claim 1, wherein tubes are coupled to the inlet of the sample reservoir and the inlet of the buffer reservoir.

4. The sensor apparatus of claim 1, wherein the carbon bundle of the working electrode is encapsulated in an insulating polymer connected to a seal.

5. The sensor apparatus of claim 1, wherein the housing a microfluidic base within which the basin is located, and elongated cavities vertically located inside the housing within which the electrodes are removably located.

6. The sensor apparatus of claim 1, wherein the reference electrode and the counter electrode are of a different material from the carbon fiber of the working electrode.

7. The sensor apparatus of claim 1, wherein the basin of a microfluidic base allows the soil and buffer mixture to flow therethrough while contacting the distal ends of the electrodes.

8. The sensor apparatus of claim 1, wherein the wall is at least one substantially horizonal cap removably fastened to an upper end of the housing, and the electrodes are removably secured to the at least one cap with proximal ends of the electrodes protruding through apertures therein.

9. The sensor apparatus of claim 1, wherein the wall is a printed circuit board which upwardly projects from the housing, and bodies of the fasteners are electrically connected to the printed circuit board.

10. The sensor apparatus of claim 1, further comprising a programmable controller configured to determine if the soil is contaminated with heavy metals including lead, mercury, cadmium and arsenic based on output from the electrodes, and when such is determined, the controller automatically determining a concentration and type of the heavy metals, and thereafter displaying the determination results.

11. The sensor apparatus of claim 1, wherein the carbon fibers of the working electrode are laser cut.

12. A sensor apparatus comprising:
a portable plate;
a housing mounted to the plate and including three cavities therein an enclosed basin therein, the basin being configured to receive a sample which operably flows therethrough;
a working electrode removably secured within one of the cavities of the housing by at least one fastener, with a distal end of the working electrode located in the basin;
a reference electrode removably secured within one of the cavities of the housing by the at least one fastener, with a distal end of the reference electrode located in the basin;
a counter electrode removably secured within one of the cavities of the housing by the at least one fastener, with a distal end of the counter electrode located in the basin;
the working electrode including a bundle of carbon fibers, each of which includes 50 to 16,300 carbon fiber strands;
at least one of the carbon fibers having a diameter of 0.94 mm to 0.28 mm;
an insulating polymer securing together the carbon fibers; and
a programmable controller automatically determining a type of heavy metal or arsenic detected in the sample.

13. The sensor apparatus of claim 12, wherein the at least one fastener removably attaches at least one substantially horizonal wall to an upper end of the housing, and the electrodes are removably secured to the at least one wall with proximal ends of the electrodes protruding through apertures therein.

14. The sensor apparatus of claim 12, wherein the at least one fastener is at least one fastener for each of the electrodes, and the at least one fastener removably attaches the electrodes to a printed circuit board which upwardly projects from the housing, and bodies of the at least one fastener are electrically connected to the printed circuit board.

15. The sensor apparatus of claim 12, wherein the reference electrode and the counting electrode are of a different material from the carbon fiber of the working electrode, and the sample is soil.

16. The sensor apparatus of claim 12, further comprising a potentiostat connecting the electrodes to a programmable controller which is in communication with a remote receiver via a wireless connection, the remote receiver displaying calculated results from output signals from at least one of the electrodes, and the sample is soil.

17. The sensor apparatus of claim 12, wherein the heavy metal or the arsenic is automatically detected in real-time by the electrodes, in the sample which includes a beverage or food, as the beverage or food flows past exposed sensing ends of the strands of the working electrode, and there are 1,450 to 16,300 of the strands in each carbon fiber of the working electrode.

18. A sensor apparatus comprising a heavy-metal sensing, working electrode further comprising:
elongated carbon fibers configured in a bundle with a laser-cut sensing end;
at least one of the carbon fibers having a diameter of 0.94 mm to 0.28 mm; and
an insulating polymer securing together the carbon fibers, each of which includes 50 to 16,300 carbon fiber strands.

19. The sensor apparatus of claim 18, wherein there are 1,450 to 16,300 of the strands in each carbon fiber of the working electrode.

20. The sensor apparatus of claim 18, wherein the carbon fiber of the working electrode includes a laser-cut sample-contacting distal end.

21. The sensor apparatus of claim 18, wherein the working, counter and reference electrodes are co-planar, with distal ends located in a cavity ending in the basin of the microfluidic base.

22. A software program for a sensor apparatus, the software program being stored in non-transient memory, the software program comprising:
a first set of instructions configured to establish a communication interface between the working electrode, reference electrode, counter electrode and a user interface;
a second set of instructions configured to send input parameters to a potentiostat;
a third set of instructions configured to energize at least one pump to flow a soil solution;
a fourth set of instructions configured to energize at least one of the electrodes and to detect electrical output data from at least one of the electrodes in contact with the soil solution;
a fifth set of instructions configured to use the potentiostat to map the data and automatically generating a graphical map on a user interface; and
a sixth set of instructions configured to automatically determine a presence of and a type of heavy metal detected.

23. The program of claim 22, wherein input parameters comprise at least one of: finale, increment, pulse width, pulse period, amplitude, quiet time, width, holding time, or holding voltage.

24. The program of claim 22, wherein calculation functions include at least one of: electrochemical impedance spectroscopy, cyclic voltammetry, differential pulse voltammetry, anodic stripping voltammetry, or combined anodic stripping voltammetry with differential pulse.

25. The program of claim 22, wherein a calculated and displayed graph is calculated in a point-by-point waveform generation up to 100 KHz.
